(19) United States
(12) Patent Application Publication
(10) Pub. No.: US 2007/0243135 A1
(43) Pub. Date: Oct. 18, 2007

(54) METHOD OF TREATING FETAL GROWTH RETARDATION AND PLACENTAL ISCHAEMIA AND INSUFFICIENCY

(76) Inventor: Debatosh Datta, Kolkata (IN)

Correspondence Address: NOTARO AND MICHALOS, 100 DUTCH HILL ROAD, SUITE 110, ORANGEBURG, NY, 10962-2100, US

(21) Appl. No.: 11/760,972

(22) Filed: Jun. 11, 2007

Related U.S. Application Data
(63) Continuation of application No. 10/294,308, filed on Nov. 14, 2002.
Application No. 10/294,308 is a continuation of application No. PCT/IN01/00105, filed on May 23, 2001.

(51) Int. Cl. A61K 31/195; A61K 38/28; A61K 49/00; A61P 43/00

(52) U.S. Cl. 424/9.1; 514/3; 514/564

(57) ABSTRACT

A method for treating intrauterine growth retardation in an animal, such as a human being, is disclosed comprising administration of a lysine composition.

### CROSS REFERENCE TO RELATED PATENT APPLICATIONS

This application is a continuation of U.S. Ser. No. 10/294,308, filed Nov. 14, 2002, which is a continuation of PCT/IN01/00105, filed May 23, 2001. The preceding applications are incorporated herein by reference in their entireties.

### FIELD AND BACKGROUND OF THE INVENTION

The invention relates generally to the treatment of fetal growth retardation in mammals with a pharmaceutical composition comprising lysine or a salt, isomer, derivative, oligomer or analogue thereof.

Intrauterine Growth Retardation (IUGR) is a problem that affects approximately three percent of all pregnancies even in the United States. IUGR is associated with significant perinatal and childhood morbidity. Infants born with IUGR are five to ten times more likely to die in the first year of life than are average gestational age (AGA) infants. Neonatal complications found to be associated with IUGR include hypoglycemia, hypothermia, hypocalcemia, polycythemia, necrotizing enterocolitis, meconium aspiration, and persistent fetal circulation. IUGR frequently appears to be due to an unexplained intrinsic disorder. It is estimated that 13.7 million infants are born annually with IUGR, comprising 11% of all births in developing countries. This higher percent is likely due to protein calorie malnutrition, and poor to no prenatal care in undeveloped countries. Public health officials recognize the urgent need for interventions aimed to prevent IUGR. There has been report giving evidence of impaired immune function in IUGR infants. IUGR infants have been reported to have fewer mature thymocytes and reduced levels of IgG, impaired lymphocyte response to mitogenic stimulation and impaired IgG production, leading to lower immunity and resistance to infections. In developing countries infants weighing 2000-2499 grams were 4 times more likely to die in the neonatal period than infants weighing 2500-2999 grams. Reduction of the incidence of IUGR can therefore be expected to reduce neonatal mortality.

Children with IUGR are at higher risk for intellectual deficit and permanent debilitating short stature. While most children with IUGR experience catch up growth within the first two years of life, 20-30% fail to grow normally. The pathophysiologic reasons determining the failure of the IUGR child to exhibit catch-up growth are unknown. IUGR has long-term effects on morbidity and mortality. Those born with IUGR have been reported to show increased incidence of cardiovascular diseases and type 2 diabetes mellitus. Experts believe that these conditions may be the result of abnormalities of endocrine development.

Sixty-five percent of IUGR pregnancies are not identified until after delivery. Early identification of IUGR is important for optimal prenatal and intrapartum medical management. IUGR is associated with a high risk of antepartum fetal death, anomalies, intrapartum hypoxia, and long-term morbidity. In order to properly manage pregnancy, labor and delivery, prenatal recognition of IUGR is important.

Insulin resistance has been reported both in childhood and adulthood in humans born after IUGR. The structures of insulin and insulin-like growth factor are similar suggesting that perhaps children born with IUGR have altered sensitivity to both.

A group of short prepubertal children born with IUGR was compared to a group of age matched controls of same height, weight, and ponderal index with normal birthweight. The two groups were tested for insulin resistance using intravenous glucose tolerance testing and tolbutamide challenge. The IUGR group demonstrated one-third the insulin sensitivity as the normal group. This supports evidence that IUGR born children are more likely to develop syndrome X (hypertension, dyslipidemia, cardiovascular disease and type two diabetes mellitus) as adults. These findings raise questions as to causation.

Could the IUGR be explained by endocrine alterations, or are the changes long-term consequences based on changes occurring in utero? Programming is a term describing the phenomenon where events occurring in early fetal development result in altered physiology. Programming has been used to explain how humans born after IUGR may suffer the adult disease states described above. The fetus adapts to a nutritionally deprived environment in utero by reducing its growth. This occurs when the endocrine milieu is altered, specifically decreased levels of IGF-I. To survive, the fetus may demonstrate wasting in order to protect the placenta. Vital organs such as the heart, brain and adrenal glands are protected with increased blood flow. The fetus responds or adapts to a decreased nutritional state by resetting hormonal regulation to a permanently slowed growth rate. After birth the infant continues its relative insulin and growth hormone resistant state and when fed a Western diet has a greater chance of developing type two diabetes mellitus. The programming theory raises questions about therapeutic administration of growth hormone, IGF-I and IGFBP-3, in the IUGR human.

The causative factors for IUGR can therefore possibly be summarized as follows:

Uteroplacental Insufficiency (80%)

maternal causes
   deficient supply of nutrients: smoking, malnutrition, multiple gestations, anemia, high altitude
   maternal vascular disease (more common): severe diabetes,
primary placental causes
   extensive placental infarctions, chronic partial separation, placenta previa
      Primary Fetal Causes (20%)
decreased intrinsic growth; symmetrical IUGR
congenital heart disease, genitourinary anomalies, CNS anomalies, chromosomal abnormalities (trisomy 13, 18, 21), viral infection (rubella, CMV)

### SUMMARY OF THE INVENTION

The analyses above provide a distinct direction towards materno-placental insufficiency in many forms. This is where a new approach can be possibly instituted in the form of induced Controlled Therapeutic Angiogenesis (CTA) where the supply insufficiency can possibly be addressed. This constitutes the principal feature of the present invention where the CTA can be induced by an essential amino acid lysine. The molecule does not have any known history of toxicity even in extremely high loading doses either through oral route or parenteral route whereas the molecule has been demonstrated to have considerable angiogenic property in different types of ischaemic tissues, e.g., cardiac, cerebral stroke, chronic wounds, etc.

The present invention provides methods for induction of angiogenesis by administration of lysine or lysine oligomers or lysine analogue. Induction of angiogenesis by the methods of the invention can be used in therapeutic angiogenesis in, for example, treatment of fetal growth disorders.

It is an object of the present invention is to provide a method of controlling, particularly enhancing angiogenesis with no adverse effects or with very limited adverse effects.

It is another object of the present invention to provide an improved method for treating fetal growth retardation or for promoting fetal growth in a mammal, comprising administering to a maternal mammalian host carrying the mammal during pregnancy an effective amount of a pharmaceutical composition comprising lysine or a salt thereof.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIGS. 1A to 1D exemplify some of the possible structures of lysine. The structure of the “native” or “ordinary” form of the essential amino acid lysine is shown in FIGS. 1A and 1B. The L- and D-enantomers of lysine are different by having mirror image orientation of the same groups and atoms around the chiral carbon atom. The “activated” form is the protonated version of the molecule, e.g., in conditions of lack of oxygen/blood supply to any tissue, including placental tissue (FIGS. 1C and 1D). Protonation can take place at the two terminal —NH₂ groups, irrespective of the —CH₂— chain length. Note that the two amino groups at the two ends of the molecule which can become protonated, e.g., in situations of ischaemia (e.g., where tissue hydrogen ion concentration goes high to various levels depending on the degree of ischaemia) and can act as the binding sites for the growth factor(s)/angiogenic factor(s) on one end and to the receptor(s) on the other end. There is a concentration window for the molecule(s) to act as the molecular bridge.

### DETAILED DESCRIPTION OF PREFERRED EMBODIMENTS

Before the present invention is described, it is to be understood that this invention is not limited to particular methodologies (e.g., modes of administration) or specific compositions described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned in this application are incorporated by the reference in their entireties to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

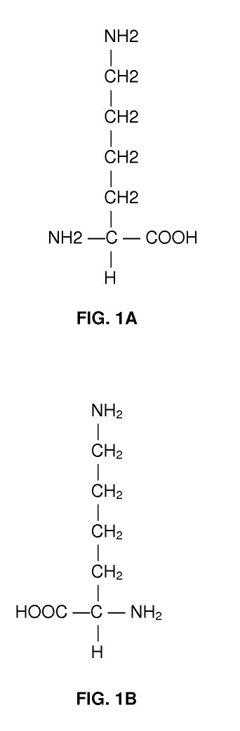

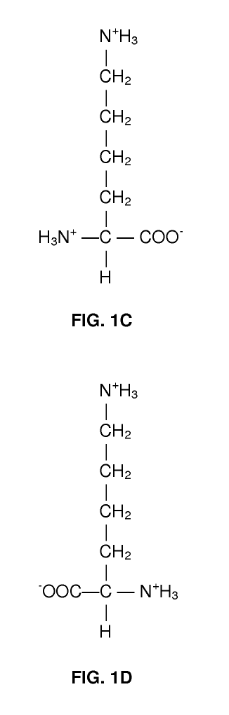

Examples of molecules or compounds which are suitable for use in a therapeutic composition of the present invention include the naturally occurring cationic essential amino acid known as lysine and its d-isomer, either in it's/their native form(s) or in it's/their “activated” form(s) where the terminal amino group(s) at each end of the molecule(s) is/are protonated preferably in tissues suffering from lack of blood supply due to various causes such as ischaemic tissues. Lysine is an off-white/white, dry powder/partly granular/amorphous solid, having a mol. wt. of about 150. It will be appreciated that lysine and its various protonated and isomeric forms may be isolated and purified from nature or synthetically prepared in any suitable manner. Some of the possible lysine structures are shown in FIGS. 1A to 1D.

Other suitable compounds include the commonly occurring salts of lysine or the various forms of lysine referred to above, such as hydrochloride, hydrobromide, hydroiodide, nitrate, sulfate or bisulfate phosphate or acid phosphate, acetate, lactate, citrate or acid citrate, tartrate, bitartrate, succinate, maleate, fumerate, gluconate, saccharate, benzoate, methanesulfonate, ethanesulfonate, benzenesulfonate, p-toluene sulfonate, camphorate and pamoate salts.

Further illustrative examples of suitable compounds include any pharmacologically acceptable derivative or metabolite of lysine or structurally similar molecule(s) which exhibit(s) pharmacotherapeutic properties similar to lysine, including its/their oligomers (e.g., mol. wt. of about 500-2500). Such derivatives, metabolites and derivatives of metabolites and structurally homologous molecules may be known in the art, and can include, but are not necessarily limited to ornithinine, putrescine, cadaverine, arginine or pharmaceutically acceptable salts thereof.

Examples of lysine derivatives includes but is not limited to d-lysine and lysine oligomers, preferably having mol. wt. of up to or around about 500 to 2500.

Further examples of lysine include its “activated” form, which is accentuated or activated in-situ after application or administration predominantly under anaerobic conditions to bring about an angiogenic response.

The terms “treatment”, “treating” and the like are used herein to generally mean obtaining a desired pharmacologic and/or physiologic effect, e.g., perfusion of the insufficiently perfused placental tissue caused by placental insufficiency. The effect may be prophylactic in terms of completely or partially preventing a disease or symptom thereof and/or may be therapeutic in terms of a partial or complete cure for a disease and/or adverse effect attributable to the disease.

“Treatment” as used herein covers any treatment of a disease in a mammal, particularly a human, and includes but is not limited to: a) preventing a disease or condition (e.g., IUGR) from occurring in a subject who may be predisposed to the disease but has not yet been diagnosed as having it; b) inhibiting the disease, e.g., arresting its development; or c) relieving the disease, i.e., causing regression of the disease (e.g., reducing fetal growth retardation or promoting fetal growth).

In the context of the present invention, stimulation of angiogenesis is employed for a subject having a disease or condition amenable to treatment by increasing vascularity and increasing blood flow. The present invention is particularly directed to the treatment fetal growth retardation in a mammalian subject by inducing controlled therapeutic angiogenesis in insufficiently perfused placental tissue through the administration of a therapeutic composition comprising lysine or a salt thereof to the mother or maternal mammalian host carrying the mammalian subject during pregnancy. In an embodiment, the mammalian subject is a fetus or a neonate in utero.

By the term effective amount, it is understood that with respect to for example angiogenic agent or angiogenic inducing agent, an amount effective to facilitate a desired therapeutic effect, e.g., a desired level of angiogenic and/or vasculogenic stimulation, is contemplated. The precise desired level of angiogenic and/or vasculogenic stimulation will vary according to the condition to be treated and/or the severity of the condition. With respect to for example the therapeutic or pharmaceutical composition, an angiogenically effective amount is contemplated.

The present invention is based on the inventor's surprising discovery that lysine, without the presence of any other angiogenic factor(s)/angiogenic growth factor(s)/vasculogenic/granulation tissue inducing agent(s), is capable of inducing angiogenesis in ischaemic tissues. The inventor's inquiries were directed towards finding new chemical additives in a high-density-culture system, in-vitro, where the objective was to expand viable hybridoma cell mass to the maximum possible at the least possible time (lag phase minimization) and maintaining the viable cell mass in culture for the maximum possible time period, in both fed-batch as well as batch) cultures. The final goal was to get highest possible yields of monoclonal antibody (Mab) in the said high-density culture. Monomeric lysine was observed to expand the biomass (cell population) at the least possible time compared to other physico-chemical means (Datta, D et al. 1997). Because of its ability to expand cells in-vitro, the cationic amino acid was taken, along with its d-isomer and short oligomer (m.w. up to or around 500-2500) to in-vivo experimentation and clinical conditions, for controlled regeneration of cells (in clinical conditions), where in-situ regeneration of cells was centrally important along with angio-neogenesis/vasculogenesis. Because the molecule has been proposed to have an indirect cell surface bridging role(s), between the cell surface receptors and the growth factor(s) (including the angiogenic factors), derived from the circulating serum protein pool, cellular expansion, migration (of the endothelial cells, forming the very early angiogenic buds) was found to be extremely rapid, reproducible and controlled (Datta, D., Unpublished results).

Prior to the inventor's disclosure, it was not known that inducing controlled angiogenesis in placental tissues could treat a fetus or infant with IUGR or at risk of developing IUGR. It was a surprising discovery that improved perfusion of inadequately perfused placental tissue through the administration of a lysine composition (or its derivative, analogue, metabolite, isomer, oligomer or salt thereof) to a pregnant mother carrying a fetus or neonate in need of treatment is a way to reduce IUGR and/or promote fetal growth.

The use of lysine or its isomer or its oligomer in a therapeutic composition of the present invention has significant advantages over the current candidates as the basis of therapeutic angiogenesis. Moreover, the pharmacology and pharmacokinetics of the essential amino acid has already been well documented and methods for systemic as well as local delivery have been investigated. Processes for the manufacture of lysine and lysine oligomers are also well characterized. Furthermore, these small molecules, particularly the l-variety (l-lysine) is synthesized easily, has no shelf-life problem, extremely stable, highly biocompatible with no known toxicity/side effects even in very high loading doses and is least costly compared to all the other currently known angiogenic agents/factors (which are all mostly proteins or peptides in their native form).

Accordingly, the invention encompasses methods and compositions for stimulation of angiogenesis and/or vasculogenesis by administration of the essential amino acid, its isomer and/or its oligomer. Of particular interest is the stimulation of angiogenesis and/or vasculogenesis in-vivo, to effect increase in blood flow, increased capillary density, and/or increased vascularity within, adjacent, or around fetal and/or maternal tissues with reduced, or lack uniform, vascular supply, including insufficiently perfused placental tissues.

Methods for production of the essential amino acid and derivatives, as mentioned, are well known in the art.

Examples of bi-amino compounds, similar structurally to the cationic amino acid of the present invention, include but are not necessarily limited to ornithine, putrescine, cadaverine, arginine, which can be provided in a herbal preparation, either in isolated form (e.g., separated or partially separated from the materials that naturally accompany the said compounds).

Additional bi-amino compounds of interest, analogous to the cationic amino acid of the present invention, can be readily identified by the ability of the candidate molecule(s) or their combinations, to stimulate angiogenesis/vasculogenesis in-vivo.

### Pharmaceutical Compositions

Upon reading the present specification, the ordinarily skilled artisan will appreciate that the pharmaceutical compositions comprising lysine and derivatives described herein can be provided in a wide variety of formulations. More particularly, the cationic amino acid can be formulated into pharmaceutical compositions by combination with appropriate pharmaceutically acceptable carriers or diluents and may be formulated into preparations in solid, semi-solid (e.g., gel), liquid or gaseous forms, such as tablet, capsules, powders, granules, ointments, solutions, suppositories, injections, inhalants and aerosols. The amino acid, being a naturally-occurring compound, can be formulated as a pharmaceutical composition or an herbal preparation.

The lysine formulation (with or without its isomer and/or oligomer) used will vary according to the condition or disease to be treated, the route of administration, the amount of the active ingredient(s) to be administered, and other variables that will be readily appreciated by the ordinarily skilled artisan. In general and as discussed in more detail below, administration of the essential amino acid/derivative(s) or formulation can be either systemic or local, and can be achieved in various ways, including but not necessarily limited to, administration by a route that is parenteral, intravenous, intra-arterial, intrapericardial, intramuscular, intraperitoneal, transdermal, transcutaneous, subdermal, intradermal, oral and intrapulmonary, etc.

In pharmaceutical dosage forms, the cationic amino acid, its derivatives or isomers, or any combination of the foregoing may be administered in the form of its/their pharmaceutically acceptable salts or it/they may also be used alone or in appropriate association, as well as in combination, with other pharmaceutically active compounds. The following methods and excipients are merely exemplary and are in no way limiting.

The amino acid/derivative(s) can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol, and if desired with conventional additives such as solubilizers, isotonic agents, suspending agents, emulsifying agents, stabilizers and preservatives.

Formulations suitable for topical, transcutaneous, and transdermal administration, e.g., to administer the amino acid/derivative directly to a desired treatment site, may be similarly prepared through use of appropriate suspending agents, solubilizers, thickening agents stabilizers and preservatives. Topical formulations may also be utilized with a means to provide continuous administration of the amino acid/derivative(s) by, for example, incorporation into slow release pellets or controlled-release patches.

The amino acid/derivative can also be formulated in a biocompatible gel, which gel can be applied topically or implanted (e.g., to provide for sustained release of the amino acid/derivative at an internal treatment site).

For oral preparations the amino acid/derivative(s) can be used alone or in combination with appropriate additives to make tablets, powders, granules, capsules for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The amino acid/derivatives can be utilized in aerosol formulation to be administered via inhalation. The compounds of the present invention can be formulated into pressurized acceptable propellants such as dichlorodifluoromethane, propane, nitrogen and the like.

Furthermore, the amino acid and/or derivative(s) can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs and suspensions may be provided wherein each dosage unit, for example, teaspoonful, tablespoonful, tablet or suppository, contains a predetermined amount of the composition containing one or more active ingredients. Similarly, unit dosage forms for injection or intravenous administration may comprise the amino acid derivative(s) in a composition as a solution in sterile water, normal saline or another pharmaceutically acceptable carrier.

The term “unit dosage form”, as used herein, refers to physically discrete units suitable as unitary dosages for human and/or animal subjects, each unit containing a predetermined quantity of the amino acid/derivative(s) calculated in an amount sufficient to produce the desired angiogenic and/or vasculogenic effect in association with a pharmaceutically acceptable diluent, carrier or vehicle. The specifications for the unit dosage forms of the present invention depend on the particular compound employed and the effect to be achieved, and the pharmacodynamics associated with each compound in the host.

The pharmaceutically acceptable excipients such as vehicles, adjuvants, carriers or diluents are readily available. Moreover pharmaceutically acceptable auxiliary substances, such as pH adjusting and buffering agents, tonicity adjusting agents, stabilizers, wetting agents and the like, are readily available.

In addition to the cationic amino acid and/or derivative(s), the pharmaceutical formulations according to the invention can comprise or be administered in parallel with agents that enhance angiogenesis by enhancing nitric oxide (NO) levels or prostacyclin levels.

Alternatively or in addition, the pharmaceutical compositions according to the invention can comprise additional angiogenesis inducing and/or vasculogenesis-inducing agents that act through other independent pathways (e.g., VEGF, FGF-a and FGF-b, etc.).

Particularly where the amino acid/derivative(s) are to be delivered for local applications, e.g., by intramuscular route, it may be desirable to provide the active ingredient(s) in a gel or matrix that can support angiogenesis, e.g., migration and proliferation of vascular cells into the matrix with endothelial tube formation. The gel or matrix can thus provide at least the initial substrate upon which new vessels form. For example, the gel or matrix can be extruded into an ischaemic region to form a path for new blood vessel formation so as to bypass an obstruction in the area.

### Induction of Angiogenesis In-Vivo

In order to accomplish stimulation of angiogenesis in vivo (e.g., as in the context of therapeutic angiogenesis), lysine and/or its derivative(s) can be administered in any suitable form/manner, preferably with pharmaceutically acceptable carrier(s). One skilled in the art will readily appreciate that a variety of suitable methods of administering lysine and/or its derivative(s), in the context of the present invention to a subject are available, and although, more than one route can be used to administer a particular compound a particular route can provide a more immediate, more effective and/or is associated with fewer side effects than another one or more routes. In general, lysine and/or its derivative(s) can be administered according to the method of the invention by, for example, a in utero, parenteral, intravenous, intra-arterial, intrapericardial, intramuscular, intraperitoneal, transdermal, transcutaneous, subdermal, intradermal or intrapulmonary route.

It is appreciated and understood that lysine and/or its derivate(s) can be administered in combination, e.g., simultaneously, sequentially or separately, with one or more therapeutically active compounds, such as growth hormone IGF-I and IGFBP-3, to enhance the growth of the fetus or infant in utero.

Local administration can be accomplished by direct injection (e.g., intramuscular injection) at the desired treatment site, by introduction of the amino acid/derivative(s) formulations intravenously at a site near a desired treatment site (e.g., into a vessel or capillary that feeds a treatment site), by intra-arterial or intra-pericardial introduction, by introduction (e.g., by injection or other method of implantation) of lysine/derivative(s) formulation(s) in a biocompatible gel or capsule within or adjacent to a treatment site, by injection directly into muscle or other tissue in which increased blood flow and/or increased vascularity is desired, by rectal introduction of the formulation(s) (e.g., in the form of suppository to, for example, facilitate vascularization of a surgically created anastomosis after resection of a piece of bowel, etc.).

In an embodiment, the lysine/derivative(s) formulation is delivered in the form of a biocompatible gel, which can be implanted (e.g., by injection into or adjacent a treatment site, by extrusion into or adjacent a tissue to be treated etc.). Gel formulations comprising lysine/derivative(s) can be designed to facilitate local release of the amino acid/derivative(s) and other active agents for a sustained period (e.g., over a period of hours, days, weeks, etc.). The gel can be injected into or near a treatment site, e.g., using a needle or other delivery device. In one embodiment, the gel is placed into or on an instrument which is inserted into the tissue and then slowly withdrawn to leave a track of gel, resulting in the stimulation of angiogenesis along the path made by the instrument. This latter method of delivery may be particularly desirable for the purpose of directing course of the bio-bypass.

In other embodiments, it may be desirable to deliver the lysine/derivative(s) formulation(s) topically, e.g., for localized delivery. Topical application can be accomplished by use of a biocompatible gel, which may be provided in the form of a patch, or by use of a cream, foam and the like. In general, topical administration is accomplished using a carrier such as a hydrophilic colloid or other material that provides a moist environment. An example of such an application would be as a sodium carboxymethyl cellulose based topical gel containing the lysine/derivative(s) and other ingredients together with preservatives and stabilizers.

In other embodiments, the lysine/derivative(s) formulation is delivered locally or systemically, preferably locally, using a transdermal patch. Several transdermal patches are well known in the art and such patches may be modified to provide for delivery of an amount of lysine/derivative(s), effective to stimulate angiogenesis according to the invention (see, e.g., U.S. Pat. Nos. 4,920,989 and 4,943,435).

In other methods of delivery, the lysine/derivative(s) can be administered using iontophoretic techniques. Methods and compositions for use in iontophoresis are well known in the art (see, e.g., U.S. Pat. Nos. 5,415,629, 5,899,876 and 5,807,306).

The desirable extent of angiogenesis will depend on the particular condition or disease being treated, as well as the stability of the patient and possible side effects. In proper doses and with suitable administration, the present invention provides for a wide range of development of blood vessels, e.g., from little development to essentially full development.

Lysine/derivative(s) based formulations mediated stimulation of angiogenic response in tissues, where reperfusion is desired, is a phenomenon which is auto regulated in-vivo. The bi-amino cationic amino acid/derivative(s) exert its angiogenic response by bridging the growth factors (angiogenic factors) to their cell surface receptors. In one embodiment, the entire process of enhancement of angiogenesis according to the present invention is entirely dependent on the availability of the amino acid/derivative(s) in a relatively high concentration in the ischaemic zone/in its immediate vicinity, as well as on the presence/availability of naturally occurring angiogenic factor(s) in the zone of ischaemia or hypoxia or in the immediate vicinity.

### Dose

The dose of lysine/derivative(s) administered to a subject, particularly a human, in the context of the present invention should be sufficient to effect a therapeutic angiogenic response in the subject over a reasonable time frame. The dose will be determined by the condition of the subject, as well as the body weight of the subject as well as the level of angiogenic growth factor(s) in an ischaemic zone/area of tissue, number of available receptor sites (for these angiogenic factors) at a given moment of ischaemia or hypoxia and the level of proton concentration ([H+]) (degree of ischaemia or hypoxia) in the ischaemic zone. The cationic amino acid being non-toxic, non-mutagenic, extremely biocompatible, loading doses have also been reported without any side effects/adverse effects.

In determining the effective amount of lysine/derivative(s) in the stimulation of angiogenesis, the route of administration, the kinetics of the release system (e.g., pill, gel or other matrix) and the potency of the active ingredient(s) is considered so as to achieve the desired angiogenic effect(s) with minimal adverse side effect(s). The amino acid formulation(s) will typically be administered to the subject being treated for a time period ranging from a day to few weeks, consistent with the clinical condition(s).

The following dosages assume that lysine is being administered or a lysine derivative with similar potency and efficiency as lysine. As will be readily apparent to the ordinarily skilled artisan, the dosage can be adjusted for lysine derivative(s) according to their potency and/or efficacy relative to lysine. If given orally, the dose may be in the range of 10 mg to 400 mg given 1 to 20 times daily and can be up to a total daily dose of about 10 mg to 8 gm. If applied topically, to provide a local angiogenic effect, the dose would likely be in the range of about 0.001 mg to 10 mg per sq. cm surface area. If injected for the purpose of a local effect, the matrix is designed to release locally an amount of cationic amino acid equivalent (l-, d-, and oligo-lysine is/are equally effective, on their own, in inducing angiogenic response) in the range of about 0.001 mg to 500 mg/unit area at the peak of activity. If injected for the purpose of a systemic effect, the matrix in which the lysine/derivative(s) is administered is designed to provide for a systemic delivery of a dose in the range of about 0.001 mg to 500 mg/dL of blood. If applied topically, for the purpose of a systemic effect, the patch or cream or gel (or any other suitable skin formulation) would be designed to provide for systemic delivery of a dose in the range of about 0.001 mg to 500 mg/dL of blood.

Regardless of the route of administration, the dose of lysine/derivative(s) can be administered over any appropriate time period, e.g., over the course of 1 to 24 hrs, over 1 to several days, etc. Furthermore, multiple doses can be administered over a selected time period. A suitable dose can be administered in suitable sub-doses per day, particularly in a prophylactic regimen. The precise treatment level will be dependent upon the response of the subject being treated. In the treatment of some individuals with lysine/derivative(s), it may be desirable to utilize a megadosing regimen. In such a treatment, a large dose of the lysine/derivative(s) is administered to an individual, and with time, the active ingredient(s) get eliminated through mainly two routes: a) rapid excretion through kidney, because of low mol. wt., and b) rapidly taken up by he cells for their metabolic needs.

### Conditions Amenable to Treatment by Lysine/Derivative(s)-Mediated Induction of Angiogenesis

The methods and lysine/derivative(s)-comprising compositions of the invention can be used to treat a variety of conditions that would benefit from stimulation of angiogenesis, stimulation of vasculogenesis, increased blood flow, and/or increased vascularity.

Examples of conditions and diseases amenable to treatment according to the method of the invention include any condition associated with inadequate oxygen and/or blood supply, such as intrauterine growth retardation.

The following examples are put forth so as to provide those of ordinary skill in the art with a complete disclosure and description of how to make and use the present invention, and are not intended to limit the scope of what the inventor regards as his invention nor are they intended to represent that the experiments below are all or the only experiments performed. Efforts have been made to ensure accuracy with respect to numbers used (e.g. amounts, temperature, etc.) but some experimental errors and deviations should be accounted for.

### EXAMPLE 1

### Oligo-Lysine and D-Lysine Induced Cell Growth and Angiogenesis In-Vitro and In-Vivo

Lysine induced repair process depends on the ability of the amino acid/derivative(s) to induce rapid and controlled cellular expansion, both in-vitro and in-vivo. The phenomenon is probably based on the ability of the molecule(s) to act as the cell surface concentrator(s) of circulating growth factor(s). The rapid angiogenic property of the molecule(s) is also entirely dependent on the same phenomenon, where angiogenic factors are concentrated, locally, on the endothelial cell surface receptors, mediated by the molecule(s) working, possibly, as molecular bridges. Oligo-lysine and d-lysine are equally effective (as l-lysine) in inducing rapid cellular growth in-vitro (FIG. 3) and angiogenesis in-vivo.

### EXAMPLE 2

### Treatment of Intrauterine Growth Retardation

Method: After diagnosis of IUGR and following admission to the antenatal ward, l-lysine HCl injection was given to patients under strict supervision, with proven growth restricted pregnancies due to placental insufficiency, as part of a controlled trial, in a dose schedule of 1 gram, 4 times a day for 7 days through intravenous route. Their PI (Pulsatility Index) and RI (Resistance Index) was measured before and after giving I.V. l-lysine HCl injection.

Result: Improved Doppler velocimetry in the umbilical artery and middle cerebral artery was observed following lysine therapy compared to control in cases of growth restricted pregnancies due to placental insufficiency. There were significant fall of PI and RI of umbilical artery in proven cases of placental insufficiency leading to IUGR compared to control.

Conclusion: L-lysine HCl injection (and possibly all other tangible salts of the amino acid, along with the d-isomer of the amino acid and or the oligomer(s) up to a mol. wt. of 2500, either in isolation or in various combination(s)), improves the perfusion of the insufficiently perfused placental tissue in cases of proven growth-restricted pregnancies due to placental insufficiency.

What is claimed is:

1. A method of reducing fetal growth retardation or of promoting fetal growth in a mammal, comprising administering to a maternal mammalian host carrying the mammal during pregnancy an effective amount of a pharmaceutical composition comprising lysine or a salt thereof.

2. The method of claim 1, wherein the mammal is a human neonate.

3. The method of claim 1, wherein the mammal has intrauterine growth retardation and/or placental insufficiency.

4. The method of claim 1, wherein the composition comprises at least one of a pharmaceutically acceptable carrier, adjuvant, diluent, additive stabilizer and excipient.

5. The method of claim 1, wherein the composition comprises at least one of l-lysine, d-lysine, activated l-lysine, activated d-lysine and oligo-lysine.

6. The method of claim 5, wherein the oligo-lysine has a molecular weight of up to about 2500.

7. The method of claim 1, wherein the maternal mammalian host is administered 1 g of lysine 4 times per day.

8. The method of claim 7, wherein lysine is administered for 7 days.

9. The method of claim 1, wherein lysine administration achieves an improved Doppler velocimetric response in umbilical artery and middle cerebral artery.

10. The method of claim 3, wherein lysine administration improves perfusion of insufficiently perfused placental tissue of the maternal mammalian host caused by placental insufficiency.

11. The method of claim 1, wherein lysine is administered to the maternal mammalian host orally or parenterally.

12. The method of claim 1, wherein lysine is administered to the maternal mammalian host intravenously, subcutaneously or intramuscularly.

13. The method of claim 1, wherein lysine is administered in utero.

14. The method of claim 1, further comprising an initial step of diagnosing growth retardation in the mammal.

15. The method of claim 14, wherein the mammal is a human fetus.

16. The method of claim 10, wherein the effective amount is an amount that achieves a desired degree of perfusion in the insufficiently perfused placental tissue.

17. The method of claim 1, wherein lysine has a structure selected from one of:

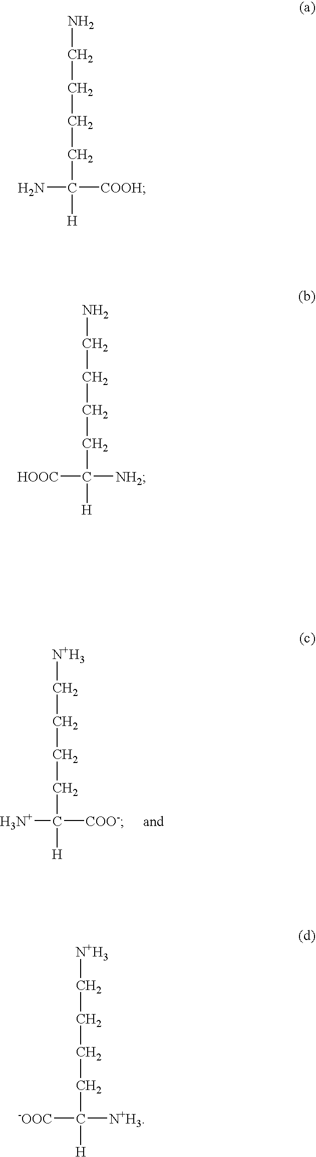

18. The method of claim 1, wherein the composition further comprises growth hormone IGF-I and IGFBP-3.

19. The method of claim 1, wherein the mammal is at risk of developing intrauterine growth retardation.
